(19) United States
(12) Patent Application Publication
(10) Pub. No.: US 2015/0366469 A1
(43) Pub. Date: Dec. 24, 2015

(54) SYSTEM FOR MEASUREMENT OF CARDIOVASCULAR HEALTH

(71) Applicant: CNV SYSTEMS LTD., Vancouver (CA)

(72) Inventors: Paul Ronald HARRIS, Vancouver (CA); Ji Feng LI, Burnaby (CA)

(21) Appl. No.: 14/650,870

(22) PCT Filed: Dec. 13, 2012
(86) PCT No.: PCT/CA2012/001151

§ 371 Date: Jun. 10, 2015

(51) Int. Cl. A61B 5/0205; A61B 5/00; A61B 5/0402

(52) U.S. Cl. CPC A61B 5/02055; A61B 5/0402; A61B 5/0022; A61B 5/6823; A61B 5/6824; A61B 5/6828; A61B 5/7207; A61B 5/7275; A61B 5/02007

(57) ABSTRACT

A system that continuously monitors cardiovascular health using an electrocardiography (ECG) source synchronized to an optical (PPG) source, without requiring invasive techniques or ongoing, large-scale external scanning procedures. The system includes an ECG signal source with electrodes contacting the skin, which generates a first set of information, and a watch-like device, worn on a limb such as an arm or a wrist, having a reflectance-based PPG signal source that generates a second set of information. Together with a processing module, housed within the watch-like device and configured to receive and process the first and second sets of information, from which the time differential of the heart beat pulmonary pressure wave can be calculated, continuous data related to cardiovascular health markers such as arterial stiffness can be determined. Automated heart rate calibration of the reflectance-based PPG sensor can also be achieved.

### PATENT REFERENCES CITED

U.S. Pat. No. 8,162,841
U.S. Pat. No. 7,674,231
US 2004/0030261
U.S. Pat. No. 7,179,228
U.S. Pat. No. 7,479,111
U.S. Pat. No. 7,481,772
U.S. Pat. No. 7,544,168
U.S. Pat. No. 7,803,120
U.S. Pat. No. 7,993,275
U.S. Pat. No. 7,029,447
U.S. Pat. No. 6,736,789
U.S. Pat. No. 6,331,162
WO 2012/092303

### OTHER REFERENCES

Boutouyrie, Pierre et al. “Obtaining arterial stiffness indices from simple arm cuff measurements: the holy grail?” Journal of Hypertension 2009, 27:2159-2161.
Conlon et al. “Development of a Mathematical Model of the Human Circulatory System” Annals of Biomedical Engineering, Vol. 34, No. 9, September 2006, pp. 1400-1413.
Douniama, C. et al. “Blood Pressure Tracking Capabilities of Pulse Transit Times in Different Arterial Segments: A Clinical Evaluation” Computers in Cardiology, 2009; 36: 201-204.
Fung et al. “Continuous Noninvasive Blood Pressure Measurement by Pulse Transit Time” Proceedings of the 26^{th} Annual International Conference of the IEEE EMBS, San Francisco, Calif., USA, Sep. 1-5, 2004.
Gesche, Heiko et al. “Continuous blood pressure measurement by using the pulse transit time: comparison to a cuff-based method” Eur J Appl Physiol, DOI 10.1007/s00421-011-1983-3, Oct. 22, 2010.
Gibbs, Peter and Asada, H. Harry “Reducing Motion Artifact in Wearable Bio-Sensors Using MEMS Accelerometers For Active Noise Cancellation” 2005 American Control Conference, Portland, Oreg., USA, Jun. 8-10, 2005.
Hey, Stefan et al. “Continuous noninvasive Pulse Transit Time Measurement for Psycho-physiological Stress Monitoring” University of Karlsruhe, House of Competence, RG hiper.campus; University of Karlsruhe, Institute for Information Processing Technology; Karlsruhe, Germany.
Kounalakis, S N and Geladas, N D “The role of pulse transit time as an index of arterial stiffness during exercise” Cardiovasc Eng. 2009 September; 9(3):92-7 Epub 2009 Aug. 6.
Liu, Yinbo and Zhang, Y. T. “Pulse Transit Time and Arterial Blood Pressure at Different Vertical Wrist Positions” Manuscript, Joint Research Centre for Biomedical Engineering Department of Electronic Engineering, The Chinese University of Hong Kong, Hong Kong, Jun. 16, 2006.
Naidu, Madireddy U R et al. “Validity and reproducibility of arterial pulse wave velocity measurement using new device with oscillometric technique: A pilot study” BioMedical Engineering OnLine 2005, 4:49 doi:10.1186/1475-925X-4-49.
Oliver, James J. and Webb, David J. “Noninvasive Assessment of Arterial Stiffness and Risk of Atherosclerotic Events” Arteriosclerosis, Thrombosis, and Vascular Biology: Journal of the American Heart Association, 2003; 23: 554-566.
Shaltis, Phillip Andrew “A Wearable Blood Pressure Sensor Using Oscillometric Photoplethysmography and Micro Accelerometers” Submitted to the Department of Mechanical Engineering in Partial Fulfillment of the Requirements for the Degree of Doctor of Philosophy in Mechanical Engineering at the Massachusetts Institute of Technology, June 2007.
Smith, Robin P. et al. “Pulse transit time: an appraisal of potential clinical applications” Thorax, 1999; 54: 452-457.

Zhang, Qiao “Cuff-Free Blood Pressure Estimation Using Signal Processing Techniques” Thesis: College of Graduate Studies and Research; Biomedical Engineering; University of Saskatchewan; August 2010.

### FIELD OF THE INVENTION

The present invention is in the field of diagnosis and monitoring of cardiovascular health.

### BACKGROUND OF THE INVENTION

Cardiovascular disease is the leading killer in the world and represents a major global health problem. It encompasses a wide gamut of disorders involving the heart and blood vessels that are typically linked. In the developed world, cardiovascular conditions such as stroke and myocardial infarction are leading killers directly caused by atherosclerosis, arterial stiffness, and hypertension.

As with any disease, prevention is far and away the better choice than post-crisis treatment, whenever possible. Annually, billions of dollars around the world could be saved in healthcare costs if cardiovascular conditions were managed before becoming life-threatening. Standards of living, particularly for the elderly, would also dramatically improve if cardiovascular conditions were caught and prevented at an early stage. To prevent cardiovascular disease, traditional diagnosis and monitoring methods have revolved around blood pressure (BP).

Conventional non-invasive blood pressure monitoring involves cuff sphygmomanometry, wherein an inflatable cuff is used to restrict blood flow, such as in the arm, and a measuring unit is used to determine at what pressure blood flow is just starting and at what pressure it is unimpeded. While relatively simple to use, cuff sphygmomanometry can be uncomfortable, and only generates one point of data at the time of use. It can also be inaccurate for various reasons, including emotional state at the time of use, time of day, and user judgment error.

Invasive means for blood pressure monitoring typically involve an intra-arterial catheter. While more accurate and capable of continuously monitoring blood pressure, invasive means run the serious risks of arterial injury and infection.

Photoplethysmography (PPG) is a modern technique for optically detecting blood volume changes in blood vessels. Typically, with transmission PPG, a photodiode emits infrared light through a small part of the body such as a finger or thumb, and that emission is detected on the other side of that small body part. Changes in light absorption are detected and can be used to measure blood flow, blood content, and other circulatory conditions.

As opposed to transmissive through an organ, a PPG sensor may instead be reflective. Reflectance PPG has significantly greater technical challenges to overcome, but the notable advantage of reflectance PPG over transmission PPG is the ability to place the emitter and the adjacent sensor to detect blood volume changes almost anywhere on the body that has blood vessels.

Many modern devices and techniques for monitoring cardiovascular health in the prior art focus on fixed BP values. However, BP in and of itself does not provide a complete measure for a particular individual's health, as BP values and health effects vary greatly between individuals. Also, it is difficult to achieve truly precise fixed systolic/diastolic values using the BP measuring devices known in the art. These devices tend to provide estimates only, and are non-continuous, momentary spot measurements that are subject to the effects of the individual's emotional and environmental circumstances at the time the measurement is taken.

Algorithms for estimating blood pressure using pulse transit time are known in the art, such as Fung et al. “Continuous Noninvasive Blood Pressure Measurement by Pulse Transit Time” Proceedings of the 26^{th} Annual International Conference of the IEEE EMBS, San Francisco, Calif., USA, Sep. 1-5, 2004.

Many doctors would agree that a more accurate indicator than a fixed BP value for any particular individual's cardiovascular health is a measure for arterial stiffness, or the elasticity of an individual's arterial walls.

Arterial stiffness has been previously measured using both invasive and non-invasive methods. Non-invasive means tend to fit into three types: 1) measuring Pulse Wave Velocity (PWV), such as with Doppler ultrasound, applanation tonometry, or MRI, 2) relating change in diameter (or area) of an artery to distending pressure, such as with ultrasound or MRI, or 3) assessing arterial pressure waveforms, such as with applanation tonometry. Such methods, and comparisons between them, can be found in Oliver, James J. and Webb, David J. “Noninvasive Assessment of Arterial Stiffness and Risk of Atherosclerotic Events” Arteriosclerosis, Thrombosis, and Vascular Biology: Journal of the American Heart Association, 2003; 23: 554-566.

Both pulse transit time (PTT) and and pulse wave velocity (PWV) have been suggested for assessment of arterial stiffness, such as in Boutouyrie, Pierre et al. “Obtaining arterial stiffness indices from simple arm cuff measurements: the holy grail?” Journal of Hypertension 2009, 27:2159-2161 and Kounalakis, S N and Geladas, N D “The role of pulse transit time as an index of arterial stiffness during exercise” Cardiovasc Eng. 2009 September; 9(3):92-7 Epub 2009 Aug. 6.

Numerous devices or systems that utilize ECG and PPG to measure BP, PTT or other cardiovascular indicators are also known in the art, such as in the following patents:

U.S. Pat. No. 7,029,447 claims a specific method and system of measuring BP using an ECG and peripheral PPG to derive pulse wave transit time.

U.S. Pat. No. 7,479,111 claims a method for measuring arterial BP through pulse transit time, using an ECG signal and a PPG signal, and compensating for other factors such as sensor contact force, nervous activities, cardiac output, and ambient temperature.

U.S. Pat. No. 7,481,772 claims a system for continuously monitoring BP through pulse transit time calculations. This system comprising a patch sensor that attaches to a patient's skin, which has an optical sensor and an electrode sensor surrounding the optical sensor, and is in communication with a separate processing component.

U.S. Pat. No. 7,674,231 claims a method of deriving an output circulatory metric, such as BP, by calculating a pulse transit time (PTT) between a first and second plethysmographic signals.

U.S. Pat. No. 7,803,120 and U.S. Pat. No. 7,993,275 claim a method and a device, respectively, for measuring BP comprising two optical sensors, two electrodes, and a microprocessor to derive three pulse transit times to determine BP.

US 2004/0030261 discloses a method and system for non-invasively measuring BP using an ECG and a peripheral PPG sensor to measure pulse wave velocity, and through that measurement estimating BP. Calibration via cuffed BP measurements is also disclosed.

U.S. Pat. No. 7,179,228 claims a device and method for measuring BP comprising a first optical module that generates a first set of information, a second optical module that generates a second set of information, an electrical sensor with an electrode pair that generates a third set of information, and a processor that calculates BP using the three sets of information.

U.S. Pat. No. 7,544,168 claims a cuff-based BP-measuring device used in conjunction with a PPG sensor. The inflatable cuff is needed for the BP measurement itself, or at the very least for calibration purposes.

U.S. Pat. No. 8,162,841 discloses and claims a non-implantable surface ECG and surface PPG system for measuring blood pressure. However, their invention entirely centres around their subcutaneously implanted device, with a simple statement that the implanted technology can be used on the surface as well. Those skilled in the art of photoplethysmography know that surface PPG has a slew of issues that must be overcome for a viable diagnostic and monitoring device to be made. Noise, accuracy, light shielding, calibration, and wearer movement are just a sampling of the issues of surface PPG that are not discussed or enabled in this patent.

U.S. Pat. No. 6,736,789 discloses and claims a blood treatment device which may comprise an ECG device and a PPG device to measure pulse transit time or pulse wave velocity.

U.S. Pat. No. 6,331,162 discloses and claims a method of measuring pulse wave velocity using two PPG probes on a patient's back, coupled with an ECG.

WO 2012/092303 discloses and claims a system for measuring stroke volume and cardiac output comprising an impedance sensor with electrodes, an optical sensor with an optical probe and optical circuit, and a processing system. The impedance sensor with electrodes is a chest-worn ECG, the optical sensor with an optical probe and optical circuit is a transmission PPG device worn on the thumb, and the processing system is a wrist-worn transceiver.

It remains highly desirable to have a means for continuous cardiovascular health monitoring, particularly for arterial stiffness, that is simple, affordable, portable, convenient, accurate, non-invasive, and compatible with modern computer and communications technology. Also, examples of current PPG devices still show the need for a simple, synchronous, portable, convenient, continuously-wearable, data-transferrable PPG sensor system.

### SUMMARY OF THE INVENTION

The present invention provides a system that continuously monitors cardiovascular health using an electrocardiography (ECG) source synchronized with a photoplethysmography (PPG) source, without requiring invasive techniques or ongoing expensive, large-scale external scanning procedures. The system includes an ECG signal source that generates a first set of information. An appendage-worn device with no external wires and a reflectance-based PPG signal source generates a second set of information. A processor, housed within the appendage-worn device, is configured to receive and process the first and second sets of information, from which the pulse transit time (PTT) of the heart beat pulmonary pressure wave can be calculated. Continuously monitored PTT can be used as a marker of cardiovascular health itself, or it can be used to calculate or estimate other cardiovascular health markers such as pulse wave velocity (PWV), blood pressure (BP), or arterial stiffness.

All of the components of the system are synchronized and function on a continuous basis over an extended period of time so as to determine an individual's relative trending markers over time and avoid the requirement for secondary calibration with outside systems. The system is designed with comfort, portability, long-term wear, and use during regular, daily-life activities in mind. There are no external wires, and the system is completely housed in comfortably worn devices that do not draw attention and do not interfere with human motion or dexterity. Such monitoring over time also allows for sustained biometric measurements, leading to clarification of an individual user's biometric signature, from which abnormalities in the rate of circulatory degeneration can be determined and applicable preventive measures applied potentially before a health crisis occurs.

The PPG source may also be coupled to an accelerometer to limit motion and signal noise. When such noise is detected to such a level as to render the PPG data defective, the synchronized system can be programmed to accept and display only accurate data from the ECG source until the PPG source is determined to be accurate again.

The invention also provides a method of use for the system to monitor, either continuously or intermittently, one or more of arterial stiffness, blood pressure, heart rate, pulse transit time, and pulse wave velocity.

### BRIEF DESCRIPTION OF THE DRAWINGS

The following figures for embodiments of the invention are meant to be read in conjunction with the detailed description:

FIG. 1 depicts a front view of a user wearing an ECG source over the heart/chest and a wrist-worn device with a PPG source on the wrist.

FIG. 2 depicts a front view of the chest strap ECG source.

FIG. 3 depicts a front view of the wrist-worn device.

FIG. 4 depicts a back view of the wrist-worn device.

FIG. 5 depicts a front perspective of the wrist-worn device.

FIG. 6 depicts a back perspective of the wrist-worn device.

FIG. 7 depicts an internal schematic of an embodiment wherein the chest strap ECG source is in wireless communication with the wrist-worn device.

FIG. 8 depicts a front view of a user wearing an embodiment wherein the ECG source consists of contact ECG electrodes on the wrist-worn device.

FIG. 9 depicts an internal schematic of an embodiment wherein the contact ECG electrodes and the PPG source are both part of the wrist-worn device.

FIG. 10 is a graph depicting a target heart beat and a target pulse wave, as detected by the ECG source and the PPG source, respectively.

FIG. 11 depicts a method of use in block diagram format.

### DETAILED DESCRIPTION OF THE PREFERRED EMBODIMENTS

### Acronyms and Definitions

For the present invention, acronyms and definitions common in the fields of cardiovascular health and mechanical engineering are to apply. Specific terms include the following:

Photoplethysmography (PPG) is the volumetric measurement of tissue using an optical device. The two common forms of PPG are transmission PPG, wherein tissue is irradiated by light and the exiting light intensity is measured by a photodetector on the other side of the tissue from the light emitter, and reflectance PPG, wherein the light emitter and photodetector are placed on the same side of the tissue and reflected light intensity is measured instead.

Pulse Oximetry is a non-invasive method, typically involving light, used to monitor the oxygenation of the blood.

Light Emitting Diode (LED) is a semiconductor light source.

Electrocardiography (ECG) is the measurement of the electrical activity of the heart, as detected by electrodes. An electrocardiogram (also ECG) is a recording of the electrical activity of the heart, as detected by electrodes attached to or contacting the skin.

Pulse Wave Velocity (PWV) is the velocity at which a pulse wave travels through the arterial tree.

Pulse Transit Time (PTT or 7) is the time it takes for a pulse wave to travel between two sites in the arterial tree.

Blood Pressure (BP or P) is the pressure exerted by circulating blood upon the walls of blood vessels. During each heartbeat, blood pressure ranges between a maximum systolic pressure when the heart contracts, and a minimum diastolic pressure when the heart is at rest.

Arterial Stiffness is the stiffness of the arterial walls.

BTLE or BLE stands for Bluetooth low energy, and is a feature of Bluetooth 4.0 wireless radio technology.

LCD stands for liquid crystal display, and is a video display that uses the light modulating properties of liquid crystals.

USB stands for Universal Serial Bus, and is an industry standard for cables, connectors, and communications protocols between computers and electronic devices.

### DESCRIPTION

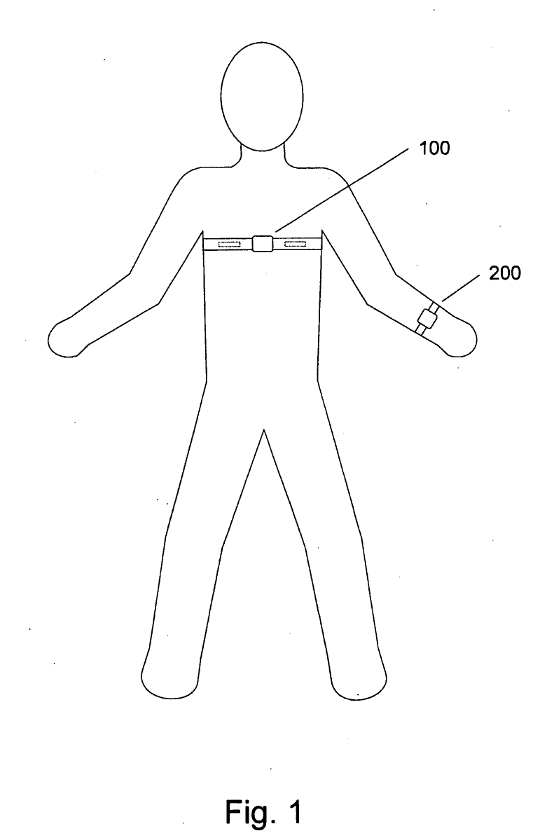

In a first embodiment of the invention, there is a chest strap ECG source (100) strapped around the chest of a person, and a wrist-worn device (200) worn around the wrist of the same person, as shown in FIG. 1.

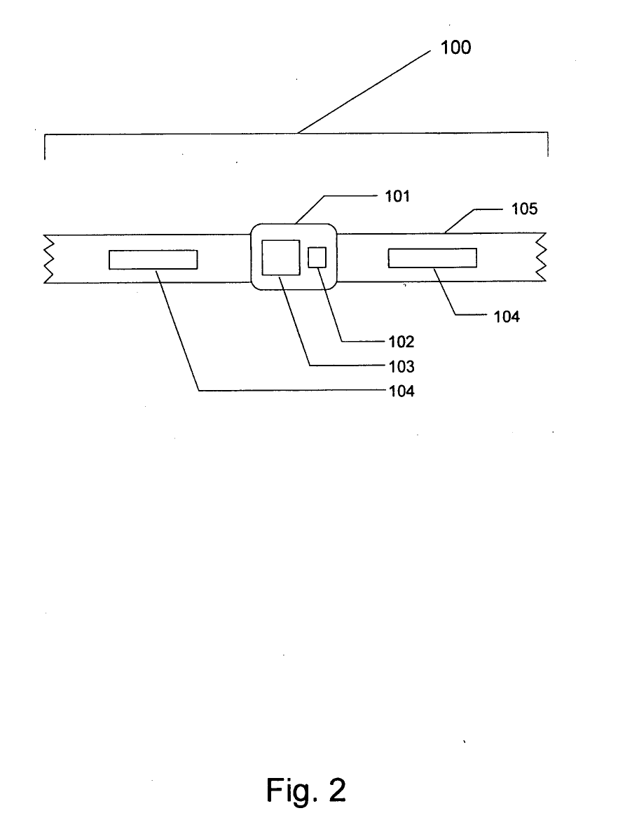

In the first embodiment, the chest strap ECG source (100) has the following components: ECG housing (101), ECG circuitry (103), elastic strap (105), wireless transmitter (102), and electrode contact strips (104), as shown in FIG. 2. The ECG circuitry (103) and wireless transmitter (102) are housed within the centrally located ECG housing (101). The elastic strap (105) extends from two opposing sides of the ECG housing (101) to strap around the wearer. When the ECG source (100) is worn, the electrode contact strips (104) on the elastic strap (105) make contact with the skin of the chest of the wearer.

Such basic ECG chest straps are known in the art and are commercially available. In the first embodiment, the ECG chest strap is modified with BTLE wireless communication.

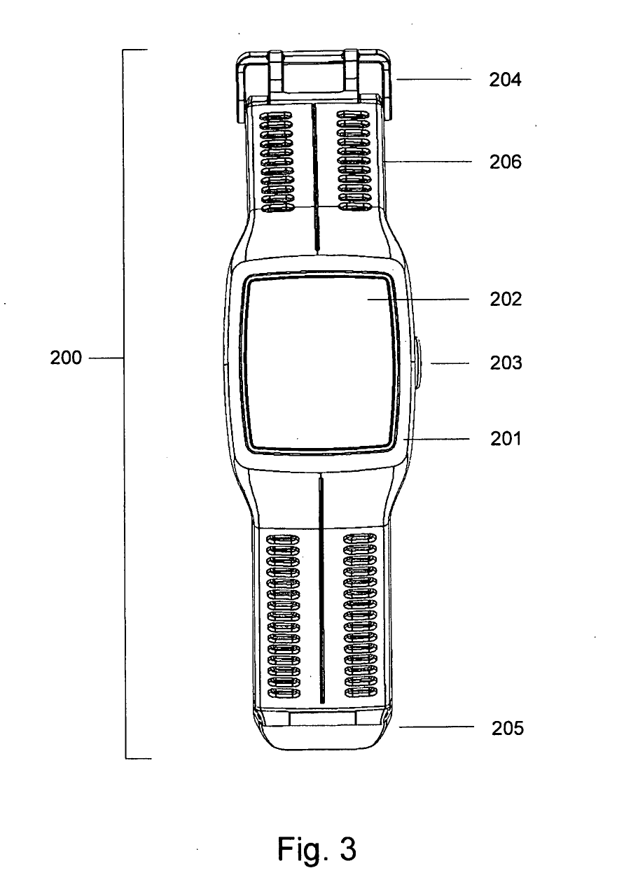

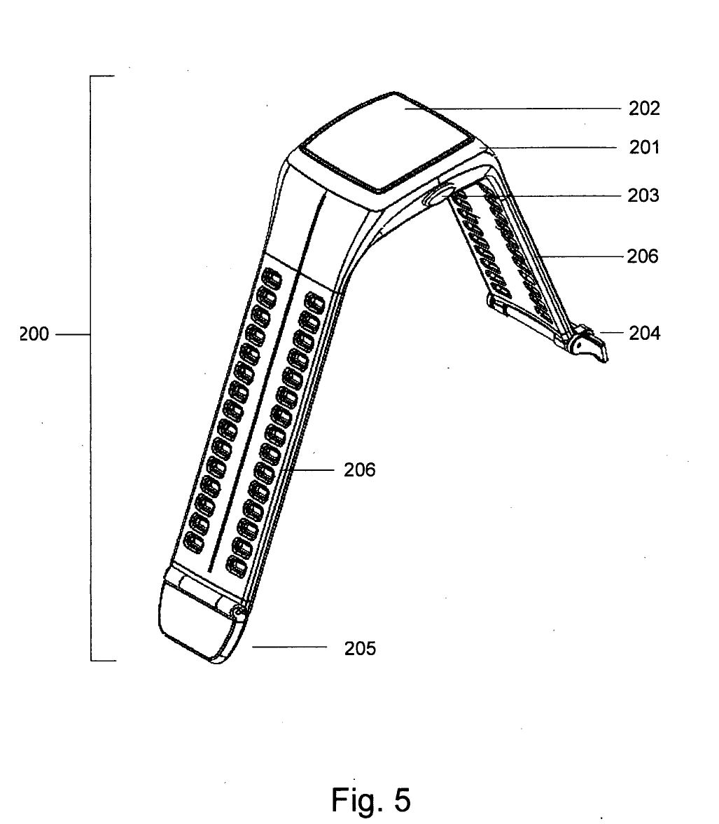

In the first embodiment, the front of the wrist-worn device (200) has the following components: housing (201), display screen (202), wristband (206), wristband clasp (204), display button (203), and USB attachment (205) as shown in FIGS. 3 and 5.

The housing (201) features the display screen (202) in the front and a display button (203) on one side. The wristband clasp (204) has two prongs that fit into the corresponding holes of the wristband (206), allowing different fits, such that the wrist-worn device (200) may be worn on any appendage. The wrist-worn device (200) also features a USB attachment (205) at one end of the wristband (206) for data transfer and battery recharge purposes.

Numerous variations of the wristband clasp (204) are possible, with one or more prongs and corresponding holes on the wristband (206). Any clasping means to secure the band around an appendage is contemplated in the present invention.

The display screen (202) is LCD in the first embodiment. It may also have touch screen functionality, in which case a display button (203) would be optional. One or more buttons on any part of the housing (201) for different displays and functions is also contemplated in the present invention. Displays and functions common in modern timepieces are contemplated herein, such as, but not limited to, time display, alarms, beepers, and stopwatch.

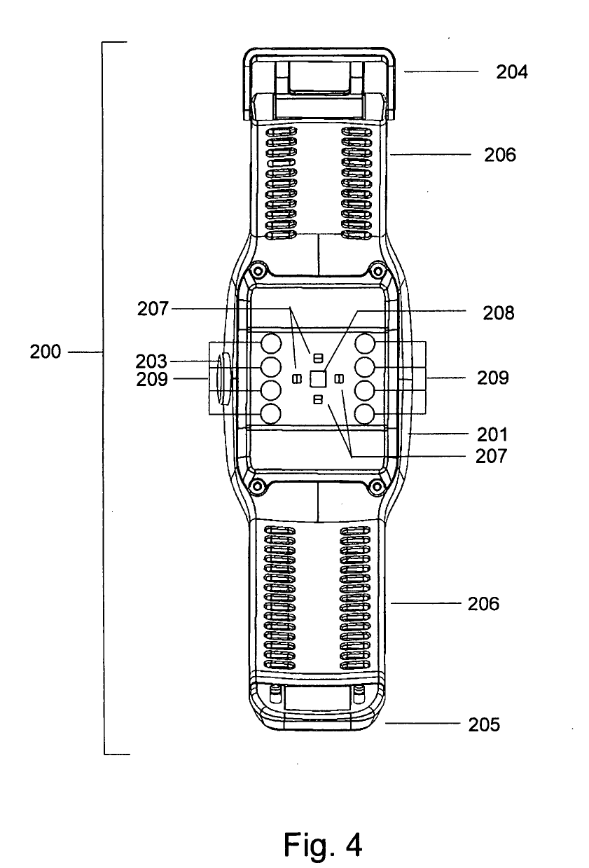

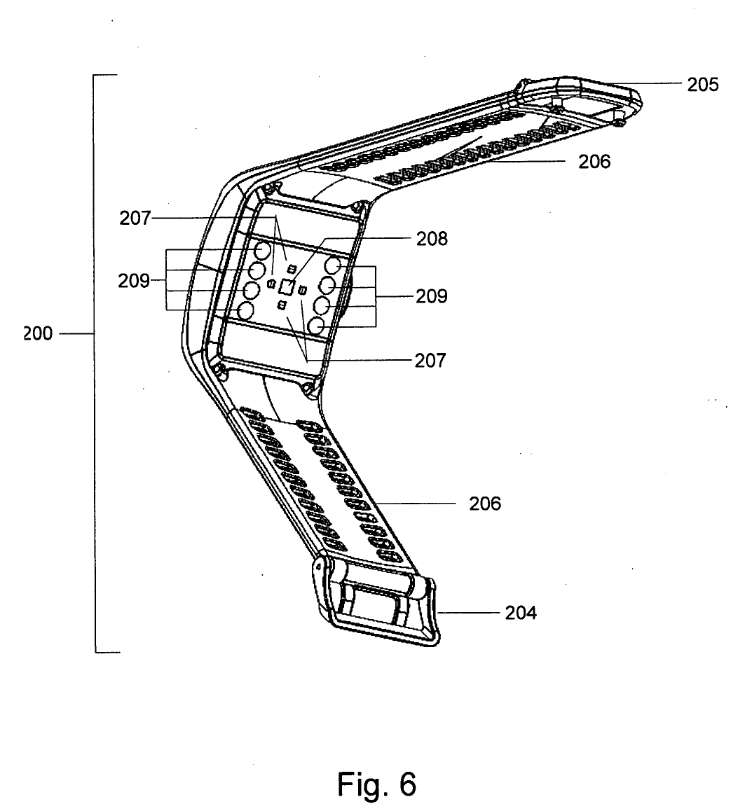

In the first embodiment, the back of the wrist-worn device (200) functions as a PPG source and has the following components: four LEDs (207) arranged equidistantly and symmetrically around an optic sensor (208), and all on the back of the housing (201) as shown in FIGS. 4 and 6. The four LEDs (207) are capable of emitting light at preferably 525 and 625 nm, although other wavelengths are possible. This light is reflected against the wearer's wrist tissue, or the tissue proximate to wherever the device (200) is worn, and detected by the optic sensor (208).

Also on the back of the wrist-worn device (200) are, eight contact sensors (209) arranged four-a-side on the back periphery of the housing (201). These contact sensors (209) press against the skin of the wearer when the device (200) is worn, and they can detect moisture and temperature. These contact sensors (209) are optional, and there may be more than or fewer than eight. They can be in any arrangement or position that allows them to make contact with the skin of the wearer when the device (200) is worn.

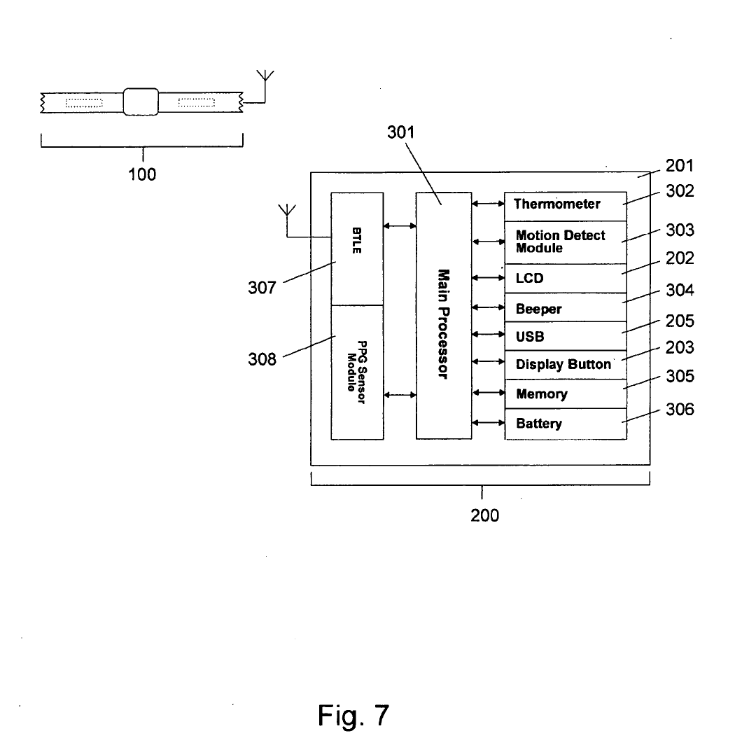

An internal schematic of the wrist-worn device (200) of the first embodiment is shown in FIG. 7, as well as the Bluetooth 4.0 wireless connectivity between the device (200) and the chest strap ECG source (100). All of the components of the wrist-worn device (200) shown are housed in the housing (201). Specifically, there is a main processor (301) which is in communication with all of the following components: a thermometer (302), a motion detect module (accelerometer) (303), the LCD display screen (202), a beeper (304), a USB attachment (205), a display input (which may be button (203) or touch screen), memory (305), a battery (306), BTLE (307), and the PPG sensor module (308) which includes the LEDs (207) and the optic sensor (208).

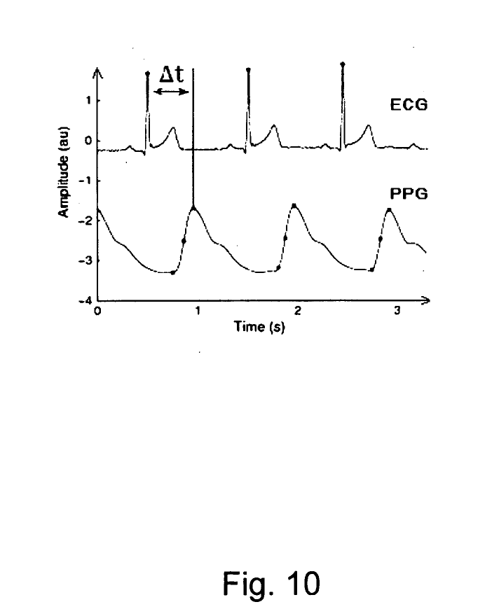

A target heart beat cycle, and its corresponding pulse wave, is shown in FIG. 10. The peak amplitude of a target heart beat is detected by the ECG. The peak amplitude of the corresponding pulse wave in the blood vessels is detected by the PPG. The difference in time between these peak amplitudes is the Δt or PTT, through which other cardiovascular health markers can be calculated. For example, PWV is the distance travelled by the pulse (which is closely approximated by the distance between the chest strap ECG and wrist-worn PPG sources, or the measured length of the artery from the heart to the wrist) divided by PTT (PWV=distance/PTT). Arterial stiffness can be derived from PWV through the Moens-Korteweg equation.

Alternatively, as seen in Zhang, Qiao “Cuff-Free Blood Pressure Estimation Using Signal Processing Techniques” Thesis: College of Graduate Studies and Research; Biomedical Engineering; University of Saskatchewan; August 2010 and Hey, Stefan et al. “Continuous noninvasive Pulse Transit Time Measurement for Psycho-physiological Stress Monitoring” University of Karlsruhe, House of Competence, RG hiper.campus; University of Karlsruhe, Institute for Information Processing Technology; Karlsruhe, Germany, other specific endpoints in the pulse waveform as detected by the PPG (three different endpoints visible in FIG. 10) may be used to determine the Δt or PTT.

Possibilities include, but are not limited to, the peak, the midpoint, the foot, the point of maximal slope, and the virtual basepoint (which corresponds to the intersection point between the tangent to the pulse wave at the point of maximal slope and the horizontal line going through the point having the absolute minimum signal). Different endpoints are suggested to have different advantages in measuring and using the PTT value. For example, using the virtual basepoint has been suggested to give a better virtual noise and artefact robustness. Using the point of maximal slope has been suggested to be strongly related to systolic BP.

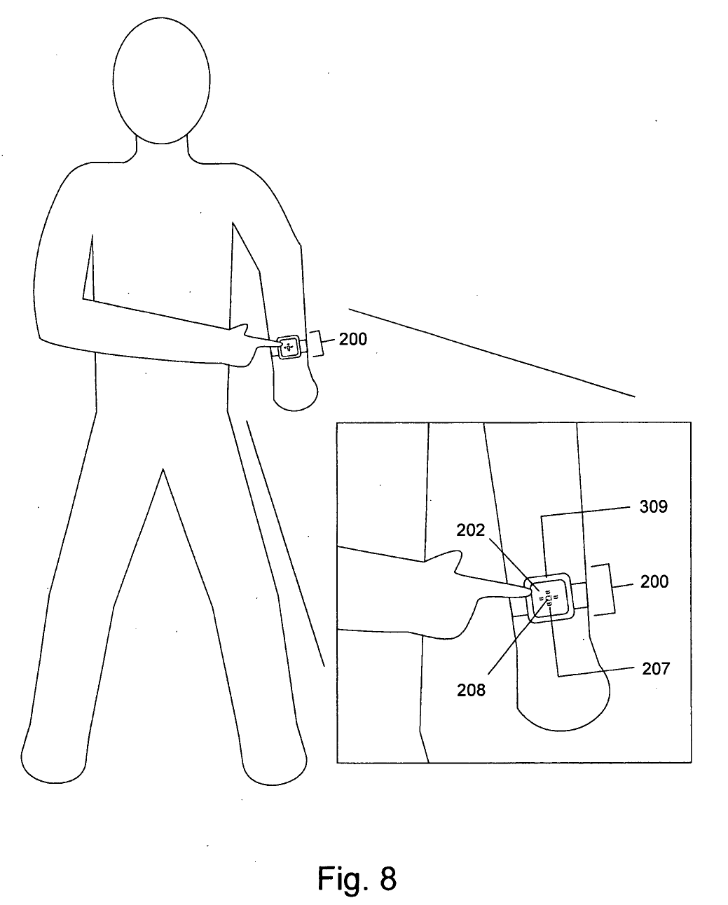

In a second embodiment, rather than a chest-worn ECG source, the ECG signal and data is obtained via two contact ECG electrodes (309) on the wrist-worn device (200) itself. As shown in FIG. 8, a first contact electrode (309) surrounds the display screen (202) on the front of the wrist-worn device (200), while a second contact electrode (not directly visible in FIG. 8) is placed on the back plating of the wrist-worn device (200) alongside the LEDs (207) and the optic sensor (208). The second contact electrode is in constant contact with the skin of the wrist while the wrist-worn device (200) is worn, while the first contact electrode (309) may be touched with a finger to complete the circuit and obtain an ECG signal and data.

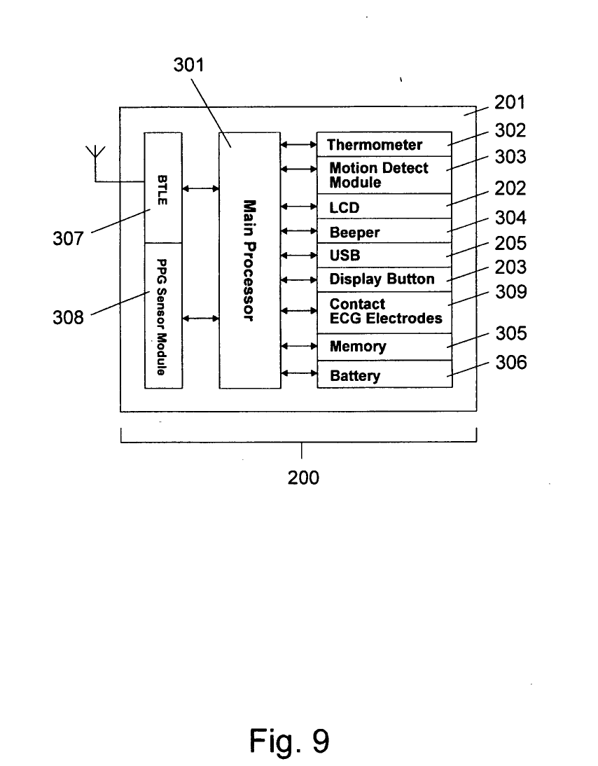

Corresponding to this second, touch ECG embodiment, an internal schematic is depicted in FIG. 9. The schematic for this touch ECG embodiment is essentially identical to the FIG. 7 schematic, except the separate chest-strap ECG source (100) is replaced by at least two contact ECG electrodes (309) in communication with the processor (301), and placed on the wrist-worn device (200) itself.

In a third embodiment, both a chest strap ECG and contact ECG electrodes on the wrist-worn device are present and used in conjunction with the PPG source on the wrist-worn device to monitor cardiovascular health. The dual ECG sources and single PPG source may be used to calibrate or correct the signal or data from each other, to more accurately determine a cardiovascular health marker such as heart rate, arterial stiffness, blood pressure, pulse transit time, or pulse wave velocity. Most preferably in this third embodiment, the heart rate signal and data for a user is continuously calibrated using this synchronized system of dual ECG and single PPG sources.

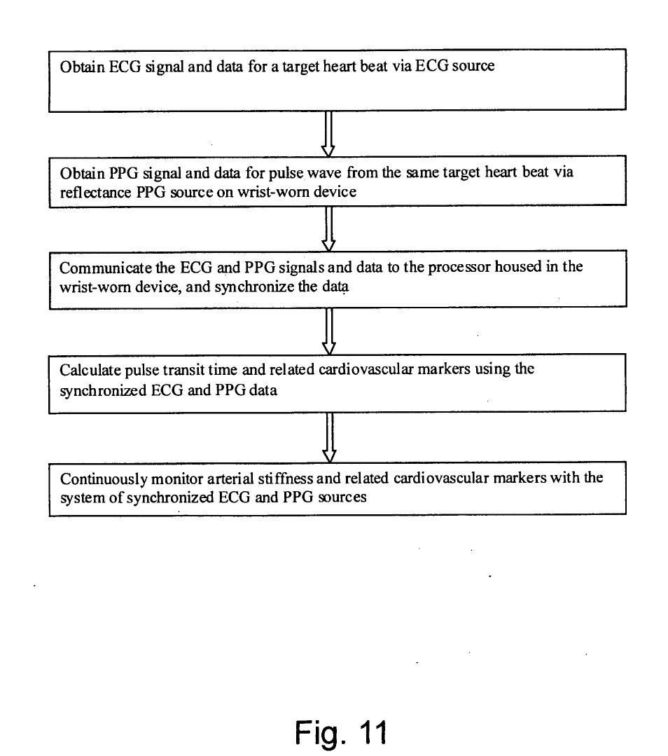

Corresponding to the system of the first embodiment, a method of use is shown in FIG. 11. To obtain a pulse transit time reading using the first embodiment, a person first straps an ECG source, having a wireless transmitter, around the chest to obtain an ECG signal and data for a target heart beat. The person then wears a wrist-worn device, having a reflectance PPG source and a wireless transceiver, on the wrist to obtain a PPG signal and data for the pulse wave in the blood vessels of the wrist caused by the same target heart beat. The ECG and PPG signals and data are communicated to a processor which is housed within the wrist-worn device. These two sets of data are synchronized by the processor and used to calculate pulse transit time and related cardiovascular indicators, such as arterial stiffness. In this manner, it becomes possible to continuously derive measurements relating to arterial stiffness and associated cardiovascular markers with this synchronized set of ECG and PPG sources, unobtrusively and without requirement for any external connecting wires. Output measurements of data stream sequences collected from subsequent date ranges can then be compared to verify cardiovascular trending markers corresponding to a specific individual's rate of arterial stiffness and circulatory degeneration, effectively providing a personalized biometric trending signature, from which preventive measures can be potentially applied before a health crisis occurs.

Optionally, an accelerometer is included with the PPG source in the wrist-worn device to help minimize motion and signal noise. A processor with the accelerometer can be used to detect when excessive noise has occurred so as to render the PPG signal and data defective. This processor then discounts the PPG data, and only the ECG data is relied upon to give cardiovascular marker information such as heart rate, until such time as the accelerometer detects that the noise has diminished and the processor determines that the PPG data is reliable again.

In the embodiment of the method shown in FIG. 11, arterial stiffness of the wearer is monitored, but the systems of the present invention can be configured to measure, calculate, or estimate one or more cardiovascular health markers over time, including, but not limited to, arterial stiffness, blood pressure, heart rate, pulse transit time, and pulse wave velocity. In another embodiment, at least two of arterial stiffness, BP, HR, PTT, and PWV are monitored over time. In another embodiment, at least three of arterial stiffness, BP, HR, PTT, and PWV are monitored over time. In another embodiment, all of arterial stiffness, BP, HR, PTT, and PWV are monitored over time. The processor can be configured to output any or all of these values at the push of a button or the touch of a display screen, simultaneously or separately.

Wireless transmitters and transceivers suitable for the present invention are known in the art, and preferably utilize Bluetooth technology, although other technologies are possible. The wrist-worn device is preferably USB compatible for computer data transfer and battery recharge purposes, although not limited to such, and other bus port types and other means for data transfer or battery recharge are possible.

In another embodiment, a light-blocking, light-filtering, or light-absorbing coating is applied to at least a portion of the back of the wrist-worn device. This coating aids in only allowing certain wavelengths of light to reach the centrally located optic sensor on the back of the wrist-worn device. This light-modifying coating is particularly useful in preventing ambient sunlight from interfering with the optical signal and data.

The LEDs of the present invention preferably emit light at 525 or 625 nm, but other wavelengths of light known in the art are suitable for reflectance PPG or pulse oximetry, and are contemplated in the present invention. In another embodiment of the invention, the PPG source on the back of the wrist-worn device can also function as a reflectance pulse oximeter, wherein the LEDs are capable of emitting a plurality of wavelengths known in the art which are suitable for either photoplethysmography or pulse oximetry. These wavelengths include, but are not limited to, 525, 625, 660, and 940 nm. This dual PPG and pulse oximetry function can be toggled via the processor, and this embodiment provides the particular advantage of having a single reflectance optical source capable of calculating any or all of the cardiovascular health markers of arterial stiffness, blood pressure, heart rate, pulse transit time, pulse wave velocity, and blood oxygen level.

In the first embodiment, the four LED wafers are specially configured to equidistantly and symmetrically surround the optic sensor so as to maintain even light distribution, reflectance, and detection. Other similar symmetrical light emitter configurations around an optic sensor are possible and are contemplated in the present invention, including less than or more than four light emitters, which may be LEDs, but not limited to such. It is also possible to surround a light emitter with a plurality of optic sensors.

The four LED wafers are each configured at a spacing of 2 mm from the optic sensor, and can be configured to any suitable angle to most ideally reflect light at the optic sensor, with the least power consumption. Various spacings and angles are possible.

Accelerometry to minimize motion and signal noise is known in the art, such as in Gibbs, Peter and Asada, H. Harry “Reducing Motion Artifact in Wearable Bio-Sensors Using MEMS Accelerometers For Active Noise Cancellation” 2005 American Control Conference, Portland, Oreg., USA, Jun. 8-10, 2005.

Combined signal measurement and data interpolation derived from protracted sequences of continuous monitoring output via the present embodiments negates or offsets the need for secondary calibration with an outside source, such as from a cuff, and it allows for determination of arterial health trending markers over time. Data derived from a continuous measurement process provides for more complete analysis of cardiovascular health indicators beyond intermittent measurements such as BP, enabling derivation of individual biometric trending that can account for anomalous BP, PTT, or PWV values due to moments of stress and other health and environmental triggers.

Continuous, unobtrusive monitoring in the present embodiments also has strong application for telemedicine purposes. Without limitation, these monitoring embodiment methods could be used to remotely validate rehabilitation compliance or fitness goals. It could be used in the doctor's office, in the hospital, in the home, and as the individual carries out daily activities. It has potential for use not only in the medical and fitness fields, but also for monitoring purposes in health insurance, policing, athletics, and military defence. It could be used to remotely store or selectively display cardiovascular health data about one or more individuals over a period of time, including during healthy or illness stages, and in determining health marker changes due to disease or aging. Those of ordinary skill in the art could identify a range of practical uses for the present embodiments.

With the sustained synchronous and continuous nature of the present embodiments, combined with the unique configuration of the LEDs in reflectance PPG, there is potential for more accurate PPG measurement of arterial performance efficiency, as opposed to the arterial measurement of the prior art.

The systems of the present embodiments offer greater usage convenience and wearability comfort than is seen with cardiovascular health monitoring devices known in the art. The present systems are designed with unobtrusive, continuous daily use in mind, whether it be in combination with a hidden chest strap ECG and wrist-worn PPG, or simply with the stand alone wrist-worn device encompassing both contact electrodes and a PPG source. The present systems do not interfere with everyday activities, as wired systems, larger systems, or finger-covering transmissive PPG systems do. The present systems are easily strapped around either the chest or an appendage, or both, in a secure, comfortable, and unobtrusive manner.

The reflectance PPG aspect is of particular benefit for unobtrusive, extended daily and night-time wear, as well as for continuous measurement and data output. However, reflectance PPG is also significantly more difficult than transmissive PPG to achieve accurate continuous readings, especially with a small, single, wearable device, which the present invention accomplishes. A major advantage of the present invention is the ability to continuously monitor cardiovascular health while wearing, at most, two discreet, comfortable, unobtrusive devices (chest strap ECG and appendage-worn reflectance PPG without wired tethering such as with extraneous transmissive PPG sensors). An even greater advantage arises with the present invention when the appendage-worn device comprises both contact ECG electrodes and the reflectance PPG source, such that all of the components needed to continuously monitor cardiovascular health are conveniently, comfortably, and unobtrusively housed in a single, compact device, at a single point on the body.

Links have been suggested throughout the prior art between the values of PTT, PWV, changes in BP, and arterial stiffness. It is generally accepted that both PTT and PWV can be regarded as indices of arterial stiffness, and that both can also be employed as estimators of BP. HR is easily monitored with either the ECG or PPG sources by measuring beats per unit time (typically beats per minute, or bpm). The present invention may be configured to calculate or estimate any or all of arterial stiffness, BP, HR, PTT, and PWV through continuous monitoring.

Much of the prior art is focussed on trying to obtain a fixed BP value for an individual to determine their cardiovascular health. More preferable than trying to calculate only a fixed BP value, though, is to determine the degeneration of arterial elasticity over time. In one embodiment, once pulse transit time is calculated, and pulse wave velocity is derived, a suitable formula for linking pulse wave velocity and arterial stiffness is the Moens-Korteweg equation:

PWV=√[(E_{inc}·h)/(2rρ)].

The Moens-Korteweg equation states that PWV is proportional to the square root of the incremental elastic modulus, (E_{inc}), of the vessel wall given constant ratio of wall thickness, h, to vessel radius, r, and blood density, ρ, assuming that the artery wall is isotropic and experiences isovolumetric change with pulse pressure.

Because of the constant ratio of wall thickness, h, to vessel radius, r, and blood density, ρ, PWV can be used as a direct correlation to arterial stiffness. With monitoring over time, changes in an individual's PWV can be directly linked to changes in arterial stiffness.

Alternately phrased, the Moens-Korteweg equation can state as follows:

PWV=√[(tE)/(ρd)],

where t is vessel wall thickness, ρ is blood density, d is the interior diameter of the vessel. As previously stated, PWV also equals the length of the vessel (L) travelled by the pulse divided by the PTT (7):

PWV=L/T.

The elastic modulus, E, is indicated as:

E=E₀e^{aP},

wherein E₀ is the modulus at zero pressure, a is dependent on the vessel, and P is the blood pressure. Making the appropriate combinations and substitutions into the Moens-Korteweg equation yields:

L/T=√[(tE₀e^{aP})/(ρd)],

which leads to:

P=(1/a)[In(L²ρd/tE₀)−(2InT)].

If changes to wall thickness t and diameter of the vessel d with respect to changes to blood pressure P are negligible, and the change in the modulus E₀ is slow enough, the change in blood pressure can be linearly related to the change in PTT as follows:

ΔP=(−2/aT)ΔT.

Similarly, through the Bramwell-Hill equation:

PWV=√[(ΔP·V)/(ρ·ΔV)],

where V is blood volume, ΔV is change in blood volume, ΔP is change in blood pressure, and ρ is blood density.

As found in the prior art through both the Moens-Korteweg and Bramwell-Hill equations, both PWV and PTT have been established to have approximate linear relationships to systolic and diastolic or mean blood pressure (P), according to the following equations:

P=(1/a)(PWV−b), and

P=(1/n)(m−PTT),

where a, b, m, and n are user or patient-specific constants.

The preferred embodiments given are meant as examples of the invention only, and not to unduly limit its scope. Those of ordinary skill in the arts of cardiovascular health and mechanical engineering will recognize that numerous variations are possible without escaping the inventive scope of the present invention.

The scope of the invention is captured by the following claims:

1. A system for monitoring cardiovascular health comprising:
a) an electrode-based module comprising at least two electrodes which contact skin, to generate a first set of data measuring a target heart beat;
b) an appendage-worn device with no external wires comprising:
i) a housing with a front and a back, the back being proximate to the skin of the appendage when the device is worn;
ii) an optical module for radiant reflective light measurement, on the back of the housing, to generate a second set of data measuring a pulse wave created by the same target heart beat corresponding to the first set of data, as the pulse wave crosses the tissue of the appendage proximate to the appendage-worn device;
iii) a processor within the housing, in communication with both the electrode-based module and the optical module, that receives the first and second sets of data and calculates at least one cardiovascular health value using the data; and
iv) a wireless transceiver, operably connected to the processor, for data exchange.

2. The system of claim 1, wherein the cardiovascular health value is at least one of arterial stiffness, blood pressure, heart rate, pulse transit time, and pulse wave velocity.

3. The system of claim 1, wherein the wireless transceiver utilizes Bluetooth technology.

4. The system of claim 1, wherein the electrode-based module is a chest-worn electrocardiographic source with a wireless transmitter.

5. The system of claim 1, wherein the electrode-based module is a contact electrocardiographic source on the appendage-worn device, said contact electrocardiographic source being in communication with the processor.

6. The system of claim 5, wherein the contact electrocardiographic source comprises:
a) a first contact electrode on the front of the housing; and
b) a second contact electrode on the back of the housing.

7. The system of claim 1, wherein there are two electrode-based modules, one of which is a chest-worn electrocardiographic source with a wireless transmitter, and the other of which is a contact electrocardiographic source on the appendage-worn device.

8. The system of claim 1, further comprising a display screen on the front of the housing which can display a cardiovascular health value calculated by the processor.

9. The system of claim 1, wherein the appendage is one of the arm, wrist, ankle and leg.

10. The system of claim 1, wherein the optical module is a reflectance photoplethysmographic source.

11. The system of claim 10, wherein the photoplethysmographic source is capable of operating at a plurality of wavelengths.

12. The system of claim 11, wherein the plurality of wavelengths comprise 525 nm and 625 nm.

13. The system of claim 10, wherein the photoplethysmographic source is configured so that four light-emitting diodes symmetrically surround a reflectance photodetector.

14. The system of claim 10, wherein at least a portion of the back of the housing is coated with a light-modifying coating.

15. The system of claim 1, wherein the optical module is both a reflectance photoplethysmographic source and a reflectance pulse oximeter.

16. The system of claim 1, wherein the appendage-worn device further comprises a bus port in communication with the processor.

17. The system of claim 1, wherein the appendage-worn device further comprises an accelerometer within the housing and in communication with the processor.

18. The system of claim 17, wherein the processor is programmed to calculate a cardiovascular health value corresponding to only one set of data should a defect be detected in the other set of data.

19. The system of claim 18, wherein the defect detected is signal noise under motion.

20. The system of claim 1, wherein the appendage-worn device further comprises at least one contact sensor, which can detect moisture and temperature, on the back of the housing.

21. A method of monitoring at least one cardiovascular health marker over time comprising the following steps, in interchangeable order where possible:
a. obtaining an ECG signal for a target heart beat to generate a first set of data;
b. obtaining a PPG signal for a pulse wave from the same target heart beat, using an appendage-worn device with no external wires and comprising a reflectance PPG source, to generate a second set of data;
c. transmitting the first and second sets of data to a processor housed in the appendage-worn device;
d. calculating at least one cardiovascular health marker with the processor, using the first and second sets of data;
e. monitoring at least one cardiovascular health marker continuously over time.

22. The method of claim 21, wherein the cardiovascular health marker is at least one of arterial stiffness, blood pressure, heart rate, pulse transit time, and pulse wave velocity.

23. The method of claim 21, wherein the ECG signal is obtained via a wireless ECG source strapped around a user's chest.

24. The method of claim 21, wherein the ECG signal is obtained via ECG contact electrodes on the appendage-worn device.

25. The method of claim 21, further comprising a step wherein a second ECG signal for a target heart beat is obtained to generate a third set of data that is also transmitted to the processor and may be used to calibrate the first and second sets of data, with a synchronized system of two ECG and one PPG sources.

26. The method of claim 21, wherein the appendage is one of the wrist, arm, ankle, and leg.

27. The method of claim 21, further comprising a step wherein the processor calculates a cardiovascular health marker corresponding to only one set of data should a defect be detected in the other set of data by an accelerometer housed with and in communication with the processor.

28. The method of claim 27, wherein the defect detected is signal noise under motion.

29. The method of claim 21, further comprising a step wherein streams of prolonged uninterrupted data measurement are collected across independent sequential date ranges and compared with each other to determine rate of change in at least one cardiovascular health marker.

30. The method of 29, wherein the streams of prolonged uninterrupted data measurement are of at least 24 hours, for each data stream.

31. A kit comprising:
a. the system of claim 1; and
b. instructions.
